(19) United States
(12) Patent Application Publication
(10) Pub. No.: US 2014/0309529 A1
(43) Pub. Date: Oct. 16, 2014

(54) RADIATION DETECTION PROBE

(71) Applicant: Dilon Technologies, Inc., Newport News, VA (US)

(72) Inventors: Michael Edward O'Neill, Randolph, MA (US); Paul Edward Glynn, Braintree, MA (US); Mamadou Diagne, Malden, MA (US); Alberto Pierotti, Cambridge, MA (US)

(21) Appl. No.: 14/163,334

(22) Filed: Jan. 24, 2014

Related U.S. Application Data
(60) Provisional application No. 61/757,057, filed on Jan. 25, 2013.

(51) Int. Cl. A61B 6/00; A61B 19/00

(52) U.S. Cl. CPC A61B 6/42; A61B 19/56
USPC 600/436

(57) ABSTRACT

Radiation detection probes for use with remote surgical robots are described. Probes may include a radiation detector (e.g., scintillator) that emits a signal (e.g., light) upon exposure to ionizing radiation originating from a radionuclide located within the body of a patient. In some embodiments, an optical fiber cable extending from a scintillator may transmit the signal to a photomultiplier and/or other signal processor, so as to ultimately provide an indication to an observer as to the potential presence/location of the radionuclide. The radiation detector may be located at a distal end of the probe, arranged to be inserted within the body of a patient during surgery, without any electrical component(s). That is, any electrical component(s) of or connected to the probe remain outside the body of the patient during surgery. The radiation detector may further be able to be inserted within the lumen of a relatively small trocar, e.g., less than 16 mm in diameter, less than 12 mm in diameter, or less than 10 mm in diameter.

### BACKGROUND

1. Field

Described herein are radiation detection probes for use in medical procedures, their construction and methods of use.

2. Discussion of Related Art

Radionuclides are atoms having an unstable nucleus that undergoes radioactive decay, resulting in ionizing radiation, which may include gamma rays, beta particle emission, etc. Radionuclides are often used in the medical field for detection of certain types of tissue. For example, in preparation for a surgical procedure, a radionuclide may be injected into a particular region of a patient where it binds or otherwise adheres to a target tissue. During the surgical procedure, an appropriate probe may be used to detect emissions originating from a radionuclide and, as a result, may indicate a region of interest to the medical team.

Certain types of radionuclides are particularly useful to determine the location of cancerous tissue within the body. In some cases, radionuclide detection techniques may be used to determine whether a cancer has spread to the lymphatic system. For example, a radionuclide is injected at the site of a tumor and, after a period of time, a detection probe is used to determine the location of the radionuclide and whether it has migrated. In breast cancer, doctors will often look for the sentinel node(s), which is the first lymph node or group of nodes through which the cancerous tissue/cells drain(s). To locate the sentinel node(s), a lymphotropic radionuclide tracer (e.g., Technetium-99) is injected into peritumoral area around the tumor, typically before performing a mastectomy or lumpectomy. As the tracer travels along the same path to the lymph nodes that the cancer cells would take, the doctor can determine the likelihood of whether the cancerous cells have migrated to other locations of the body.

Surgical robots have enabled doctors to operate on patients with minimal invasiveness, in part, so as to allow for a relatively quick recovery. A surgical robot system may include one or more arms and instruments that are remotely controlled by a trained operator. While, in several cases, surgical robots are controlled by surgeons that are located within the same room or building within which the surgery is taking place, such systems may also allow for surgeons to perform operations on patients from distant locations, making the expertise of specialized doctors available to patients worldwide.

### SUMMARY

The inventors have appreciated that it would be beneficial to construct a radiation detection probe that may be suitably used with a surgical robot, for carrying out medical procedures (e.g., related to cancer surgery). The detection probe may be configured to indicate the presence of a radiation source, such as a radionuclide, located within a patient. Accordingly, a surgical robot may be controlled by a user to grasp and manipulate the probe in a precise manner, so as to determine the presence/location of the radiation source within the patient. In some cases, a surgical robot may allow a user to operate on a patient from a remote location.

The probe may include a radiation detector (e.g., scintillator, crystal, radiation counter, etc.) that emits a signal (e.g., optical signal) upon exposure to ionizing radiation originating from a radionuclide. The radiation detector may be located at a distal end of the probe and arranged so as to be inserted within the body of a patient during the medical procedure. The radiation detector may further be constructed so that it is able to be inserted within the lumen of a relatively small trocar (e.g., less than 16 mm in diameter, less than 12 mm in diameter, less than 10 mm in diameter), for appropriate manipulation within the patient during surgery.

A signaling cable (e.g., optical fiber cable) may be connected to the radiation detector for transmitting the signal emitted from the radiation detector toward a proximal end of the probe. The cable may transmit the signal from the radiation detector toward a signal processor and/or controller, for subsequent processing of the signal emitted from the radiation detector. For some embodiments, when the signal emitted from the radiation detector includes light, the signal processor and/or controller may include a photomultiplier, for outputting an electrical current based on the amount of light received. In some embodiments, any electrical components, including the signal processor and controller, associated with the radiation detector, may be arranged to remain outside the body of the patient during the medical procedure, even though the radiation detector itself, during the medical procedure, may be inserted within the body of the patient.

In an illustrative embodiment, a probe having a distal end and a proximal end, configured for use in a medical procedure, is provided. The probe includes a scintillator constructed to be able to be inserted into a body of a patient by passing through the lumen of a trocar, wherein the lumen has a largest cross-sectional dimension not exceeding 16 mm, and wherein the scintillator is adapted to emit a signal upon exposure to a radionuclide; and an optical fiber connected to the scintillator, for transmitting the signal emitted from the scintillator.

In another illustrative embodiment, a probe having a distal end and a proximal end, configured for use in a medical procedure, is provided. The probe includes a radiation detector located at the distal end, adapted to emit a signal upon exposure to a radionuclide, wherein the radiation detector is constructed to be able to be inserted into a body of a patient by passing through the lumen of a trocar, wherein the lumen has a largest cross-sectional dimension not exceeding 16 mm; and a flexible cable connected at its distal end to the radiation detector and connected or connectable at its proximal end to a signal processor and/or controller, the flexible cable configured to transmit the signal emitted from the radiation detector and/or information generated from the signal to the signal processor and/or controller.

In a further illustrative embodiment, a probe system comprising a probe having a distal end and a proximal end, configured for use in a medical procedure is provided. The probe includes a radiation detector located at the distal end of the probe, adapted to emit a signal upon exposure to a radiation source and arranged to be inserted within the body of a patient during the medical procedure; and a signal processor and/or controller located at or connected to the proximal end of the probe, configured to process the signal emitted from the radiation detector and to remain outside the body of the patient during the medical procedure, wherein the distal end of the probe that is inserted into the body of the patient during the medical procedure is free of any electrical component.

In yet another illustrative embodiment, a system for performing a medical procedure is provided. The system includes a surgical robot configured to perform remote surgery; and a probe comprising a grasping feature shaped and arranged to facilitate being grasped and manipulated by an arm of the surgical robot, wherein the probe is configured to detect a radiation source located within a body of a patient.

In another illustrative embodiment, a method for performing a medical procedure, is provided. The method includes grasping a probe using a surgical robot, wherein the probe is configured to detect a radiation source located within a body of a patient; and manipulating the probe within the body of the patient with the surgical robot to detect the radiation source within the patient.

In a further illustrative embodiment, a surgical device configured for use in a medical procedure is provided. The device includes a radiation detector adapted to emit a signal upon exposure to a radionuclide, wherein the radiation detector is constructed to be able to be inserted into a body of a patient by passing through the lumen of a trocar, wherein the lumen has a largest cross-sectional dimension not exceeding 16 mm; and wherein the radiation detector comprises a connector configured for connection to a flexible cable for transmitting the signal emitted from the radiation detector and/or information generated from the signal to a signal processor and/or controller, and/or comprises a transmitter configured to wirelessly transmit the signal emitted from the radiation detector and/or information generated from the signal to a signal processor and/or controller.

In another illustrative embodiment, a surgical device for robotic surgery is provided. The device includes a probe comprising a grasping feature shaped and arranged to facilitate being grasped and manipulated by an arm of the surgical robot, wherein the probe is configured to detect a radiation source located within a body of a patient.

In yet another illustrative embodiment, an apparatus having a distal end and a proximal end, for use with a surgical robot, is provided. The apparatus includes a handle having a surface that complements a surface of the arm of the surgical robot to facilitate being grasped and manipulated by the arm of the surgical robot; a scintillator located at the distal end, constructed to be able to be inserted into a body of a patient by passing through the lumen of a trocar, wherein the lumen has a largest cross-sectional dimension not exceeding 16 mm, and wherein the scintillator is adapted to emit an optical signal upon exposure to a radionuclide; a photomultiplier located at the proximal end, configured to receive the optical signal emitted from the scintillator and generate an electrical current based on an amount of optical signal received from the scintillator, the photomultiplier configured to remain outside the body of the patient during the medical procedure; a flexible optical fiber cable connected at its distal end to the scintillator and connected or connectable at its proximal end to the photomultiplier, the flexible optical fiber cable configured to transmit the optical signal emitted from the scintillator to the photomultiplier; and wherein the distal end of the apparatus that is inserted into the body of the patient during the medical procedure is free of any electrical component.

Various embodiments of the present invention provide certain advantages. Not all embodiments of the invention share the same advantages and those that do may not share them under all circumstances. Various embodiments described may be used in combination and may provide additive benefits.

Further features and advantages of the present invention, as well as the structure of various embodiments of the present invention are described in detail below with reference to the accompanying drawings.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. Various embodiments of the invention will now be described, by way of example, with reference to the accompanying drawings, in which:

FIG. 1 depicts a conventional radiation detection probe system;

FIGS. 2A-2B illustrate a radiation detection probe in accordance with some embodiments;

FIG. 3 shows a surgical robot system in accordance with some embodiments; and

FIG. 4 depicts a radiation detection probe in use with a surgical robot in accordance with some embodiments.

FIG. 5 illustrates another radiation detection probe in accordance with some embodiments.

### DETAILED DESCRIPTION

The present disclosure describes radiation detection probes (e.g., gamma uptake probes), in certain embodiments specifically constructed for use with a surgical robot. The probes may be configured, for performing a wide variety of medical procedures, especially minimally invasive surgical procedures and/or procedures performed laparoscopically and/or through percutaneous insertion of instruments into the body of a patient via, for example, a trocar or similar introducer device. In certain cases, the probes are particularly suitable and/or adapted for use in surgical procedures in which the probes are inserted into a patient and manipulated by instruments remotely, e.g. via a surgical robot system, as described in more detail below. The detection probe may be configured to detect the presence of a radiation source (e.g., gamma ray source), such as a radionuclide that had been previously injected into a patient, for example, for purposes of cancer detection. That is, in some cases, a series of radionuclides that adhere to or otherwise tag cancerous tissue/cells may be injected into a patient at the site of a tumor. If the tumor migrates, the radionuclide(s) will migrate therewith. When detected, the radiation released from the radionuclides may be useful to medical care personnel as an indicator to determine whether a cancer has spread, for example, to the lymph nodes and/or other regions of the body.

Conventional probes used to detect radionuclides are made to be handled manually, and are unable to be effectively and/or securely held and operated in an appropriate manner by a surgical robot or other remotely operated manipulation system. By contrast, probes configured in accordance with certain embodiments of the present disclosure may be suitably grasped and manipulated in a complex manner by a surgical robot or other remotely operated manipulation system. For instance, the surgical robot, controlled by a user from a remote location, may move the probe precisely along a complex spatial path, so as to assist a doctor in locating the radiation source within the body of the patient, as if the probe were handled by an actual person.

The probe may include a radiation detector (e.g., gamma ray detector) that emits a signal upon exposure to radiation released from the radionuclide. For example, as discussed further herein, the radiation detector may be a scintillator that emits visible light when exposed to ionizing radiation (e.g., gamma rays, beta particles, etc.). During use, the radiation detector, located at the distal end of the probe, is inserted and appropriately manipulated within the body of the patient, in search of the injected radionuclide.

The probe may include or be configured for connection to a cable connected or connectable to the radiation detector. The cable may extend from the radiation detector toward a proximal end of the probe where a signal processor and/or controller may be located. For example, the cable may include an optical fiber arrangement for transmitting signal (e.g., light) emitted from the radiation detector therethrough, to the signal processor and/or controller, where the signal may be suitably processed. In some embodiments, when the radiation detector emits an optical signal, such as light, the signal processor and/or controller may include a photomultiplier (e.g., solid state photomultiplier) or other appropriate device for receiving the optical signal and otherwise processing the signal by generating an electrical current based on the amount of signal received. For some embodiments, during the medical procedure, when the radiation detector is inserted and manipulated within the body of the patient, the cable may be long enough such that the signal processor and/or controller to which the cable is connected remains outside the body of the patient.

In some embodiments, the distal end of the probe that is inserted into the body of the patient during the medical procedure is free of any electrical component (e.g., transistors, diodes, power sources, semiconducting devices, etc.). For example, the distal end of the probe may include a scintillator and an optical fiber cable connected thereto for transmitting light produced by the scintillator, without further need for electrical components integrated therewith.

Conventional radiation detection probes, on the other hand, typically require the distal end of the probe that is inserted into the body of the patient during the medical procedure to include one or more electrical components. The electrical component(s) are typically connected directly to the radiation detector so as to process the signal (e.g., electrical current) emitted by the radiation detector, upon exposure to radiation released from the radionuclide, for later display.

It may be advantageous, as recognized within the context of the present invention, for the electrical component(s) to not be placed in such close proximity to the body of the patient. For instance, the electrical component(s) may give off excessive amounts of heat and/or produce undesirable current/voltage, which could ultimately damage surrounding tissue. Thus, in accordance with certain embodiments of the present disclosure, the lack of electrical components at the distal end of the probe mitigates the potentially harmful risk(s) (e.g., short circuit, current leakage, heat buildup, etc.) associated with bringing the component(s) in close proximity to living tissue.

Further, a probe incorporating electrical components at or near its distal end may be excessively large or bulky for suitable use in minimally invasive procedures and/or by procedures employing a surgical robot. For instance, the probe might not fit within the lumen of a trocar of a preferred size. Accordingly, without having to include any electrical components at the distal (insertion) end of the probe, the probe may be more easily constructed to be smaller (e.g., in diameter, width, cross-sectional area, or other dimension(s)) than would otherwise be the case if one or more electrical components are incorporated within the probe. That said, in certain embodiments, a surgical probe of the invention may use a radiation detector and/or other features involving electrical components at or near its distal end and configured for insertion into a patient during use. Such probes in certain cases may still be sized/miniaturized to facilitate suitable use in minimally invasive procedures and/or by procedures employing a surgical robot. For example, in some embodiments, the radiation detector and any other portions of the probe to be inserted into the patient during a surgical procedure may be constructed so as to be able to be inserted through the lumen of a small-sized trocar. Such a lumen may, for example, have a largest cross-sectional dimension not exceeding or essentially equal to 16 mm, in certain cases not exceeding or essentially equal to 14 mm, in certain cases not exceeding or essentially equal to 12 mm, in certain cases not exceeding or essentially equal to 10 mm, in certain cases not exceeding or essentially equal to 8 mm, and in other cases, not exceeding or essentially equal to 5 mm. As a result, the trocar may provide a port through which the probe may be fed into the body of the patient and used for its intended purpose.

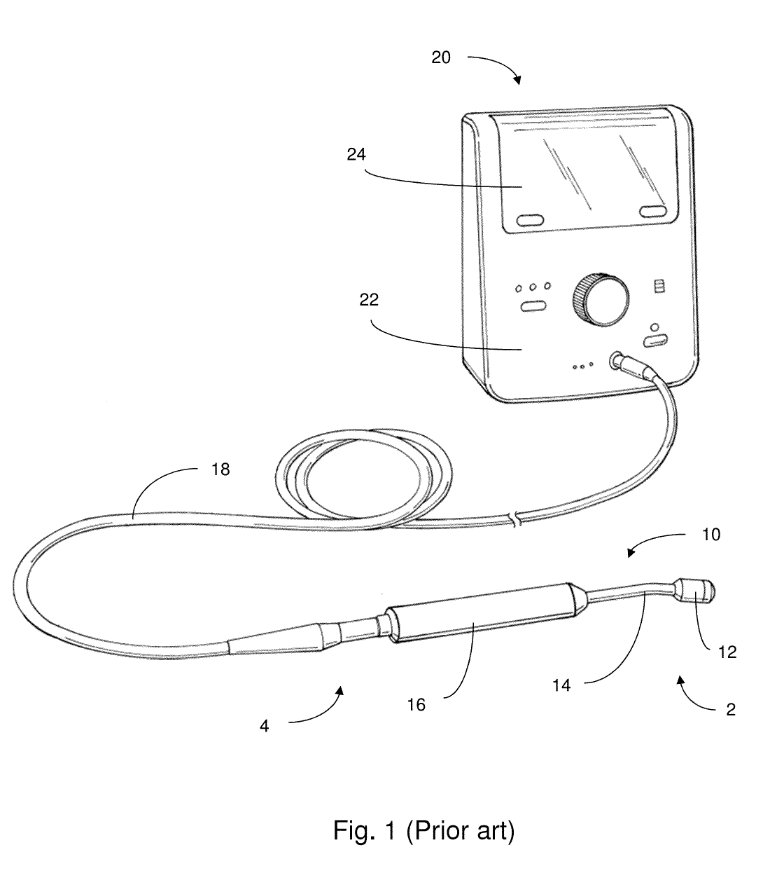

FIG. 1 depicts a conventional radiation probe system including a probe 10 and a controller 20. Here, the probe 10 is manually handled during open surgery, rather than introduced via a trocar and/or employed with surgical robots described herein, to assist a doctor in identifying the location of a radionuclide within the body of a patient. As shown in FIG. 1, the probe 10 is connected to the controller 20 via an electrical wire 18, though, the probe 10 may also be connected wirelessly to the controller 20. In general, during use, the distal end 2 of the probe is either inserted into the body of the patient or scanned across the body of the patient, for detecting the location of the radionuclide; and the proximal end 4 of the probe remains outside of the body.

The probe 10 includes a radiation detector 12, an intermediate extension 14 and a housing 16. Here, the radiation detector 12 includes a radiation detecting material, for example, a cadmium telluride (CdTe) crystal, which has a property such that, upon exposure to ionizing radiation (e.g., gamma rays, beta particles, etc.), the crystal produces an electrical current.

In general, the amount of current generated by a CdTe crystal may depend on the level(s) of ionizing radiation to which the crystal is exposed. For instance, when the crystal is located relatively far from the radionuclide, the amount of ionizing radiation exposure thereto is insubstantial, and so the amount of electrical current produced by the crystal is small, or negligible. On the other hand, when the crystal comes into close proximity to the radionuclide, the crystal is exposed to a much greater amount of ionizing radiation which, in turn, causes the crystal to generate a comparatively greater amount of electrical current. Thus, the amount of electrical current generated by the crystal may provide an indication as to whether the radionuclide is present at the particular location interrogated by the probe 10.

To measure the amount of electrical current generated by the crystal, the probe 10 requires the appropriate electrical component(s) (e.g., electrical circuitry, power source, etc.) to be present at the distal end 2, connected to the crystal.

The electrical component(s) within the probe 10 may be relatively fragile. Consequently, operators of the probe 10 must be extremely careful not to inadvertently drop the probe 10 for fear of incurring damage thereto upon impact. Accordingly, the radiation detector 12 includes a relatively large housing for protection and support of the crystal along with the various component(s) necessary to provide a suitable indication of the level of current produced by the crystal.

The extension 14 and housing 16 may include additional electrical and power components appropriate to support communication between the radiation detector 12 and the controller 20, and/or other components of the overall system. The extension 14 and housing 16 may also be constructed and shaped in a relatively large/bulky manner so as to provide for suitable manual handling of the probe 10.

The controller 20 includes a console 22 and a display screen 24. The console 22 allows a user to adjust various parameters of detection for the probe, such as selecting the isotope, volume and threshold for detection. For example, depending on the type of radionuclide that is to be detected, the amount of current generated by the crystal may vary, hence, the console 22 may be adjusted according to the particular radionuclide of interest. Accordingly, during use, the console 22 may be set to appropriate threshold levels for detecting the radionuclide of interest, and the display screen 24 may provide a visual and/or audio indication as to whether the particular radionuclide has been detected by the radiation detector.

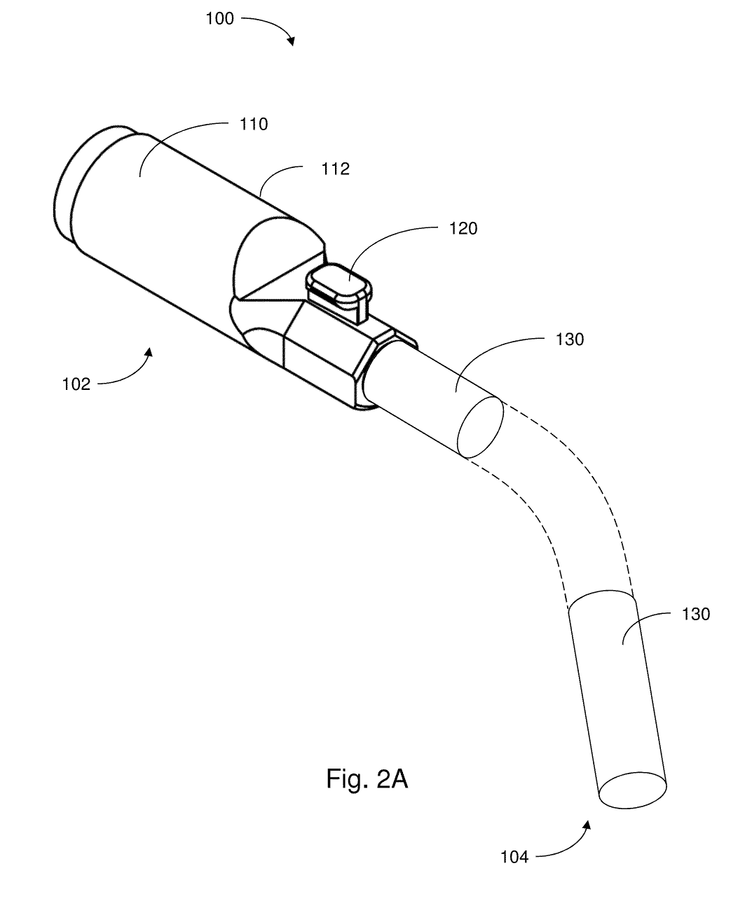

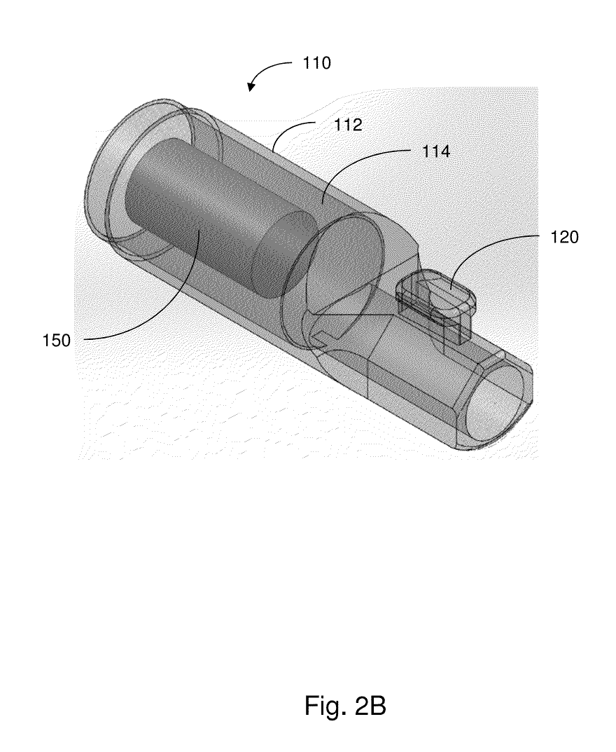

FIGS. 2A-2B illustrate a probe 100 in accordance with certain embodiments of the present disclosure. The probe 100 is constructed such that, during use in surgery, the distal end 102 of the probe is inserted into the body of the patient, while the proximal end 104 of the probe remains outside of the body of the patient. The probe 100 includes a radiation detector 110, an attachment region 120 and a cable 130. In certain embodiments, probe 100 may be configured with a wireless transmitter to facilitate wireless transmission of signal/information generated by the radiation detector to a remotely located controller and/or data processor unit, e.g. like controller 20 in FIG. 1.

FIG. 2B depicts a radiation detector 110, located at the distal end 102 of the probe 100. The radiation detector 110 includes a housing 112, a collimator 114 and a radiation detecting material/component 150 located within a space defined by the housing. For illustrative purposes, the radiation detecting material/component 150 is shown to be disposed within the collimator 114 which is, in turn, disposed within the housing 112. In some embodiments, the collimator (e.g., tungsten) may be used to shield energy emitted from the side of the detector, serving to narrow the signal toward the cable 130. The housing 112 (e.g., stainless steel) may provide a layer of protection for the components disposed therein. The radiation detecting material/component 150 may have certain properties such that, upon exposure to ionizing radiation (e.g., gamma rays, beta particles, etc.), the radiation detecting material 150 produces a signal that is emitted therefrom. In some embodiments, the intensity and/or type of the signal produced from the radiation detecting material 150 will vary according to the level of ionizing radiation to which the material is exposed.

In some embodiments, the radiation detecting material/component 150 may be any suitable radiation detecting material/component known in the art and may be the same as or similar to materials described above for the radiation detector 12 in FIG. 1, and as such may include a radiation detecting material that is, for example, a cadmium telluride (CdTe) crystal, which has a property such that, upon exposure to ionizing radiation (e.g., gamma rays, beta particles, etc.), the crystal produces an electrical current. In some preferred embodiments, the radiation detecting material/component 150 is a scintillator, which is characterized in that the scintillator luminesces when exposed to gamma radiation and/or other types of ionizing radiation or particles. Generally speaking, a scintillator will absorb energy when struck by incoming particles or radiation, and re-emits the absorbed energy outward as light. Depending on the type of scintillator, the level, or intensity, of luminescence produced will depend on the amount of a particular ionizing radiation to which the scintillator is exposed. That is, the more ionizing radiation detected by the scintillator, the greater the intensity of light is given off by the scintillator. In some embodiments, the amount of light emitted by the scintillator may be proportional (e.g., linearly, exponentially, etc.) to the amount of ionizing radiation to which the scintillator is exposed. While in various embodiments of the present disclosure, the radiation detecting material/component is a scintillator, it can be appreciated that other radiation detecting materials/components may be used, for example, a radiation detecting crystal, a Geiger-type tube, a semiconductor radiation detector, amongst others.

The scintillator may include any suitable material. In some embodiments, the scintillator includes a bismuth germinate (BGO) crystal, or a similar material that provides a desirable signal to noise ratio for a particular radionuclide of interest, without significant delay. Depending on the amount and type of ionizing radiation emitted, which is governed by the type of radionuclide located within the body, other scintillators may be used. Scintillators that may be used as radiation detecting materials described herein may include BGO, barium fluoride, calcium fluoride optionally doped with europium, cadmium tungstate, calcium tungstate, cesium iodide optionally doped with thallium, cesium iodide optionally doped with sodium, lanthanum bromide optionally doped with cerium, lanthanum chloride optionally doped with cerium, lead tungstate, lutetium iodide, lutetium oxyorthosilicate, lutetium yttrium orthosilicate, sodium iodide optionally doped with thallium, yttrium aluminum garnet optionally doped with cerium, zinc sulfide optionally doped with silver, zinc tungstate, or any other material that exhibits suitable scintillation behavior. It can be appreciated that for some embodiments, the radiation detecting material 150 is made up of a composition other than a scintillator, for example, a material that emits another type of signal (e.g., audio, visual, electromagnetic, electrical, etc.) upon exposure to ionizing radiation.

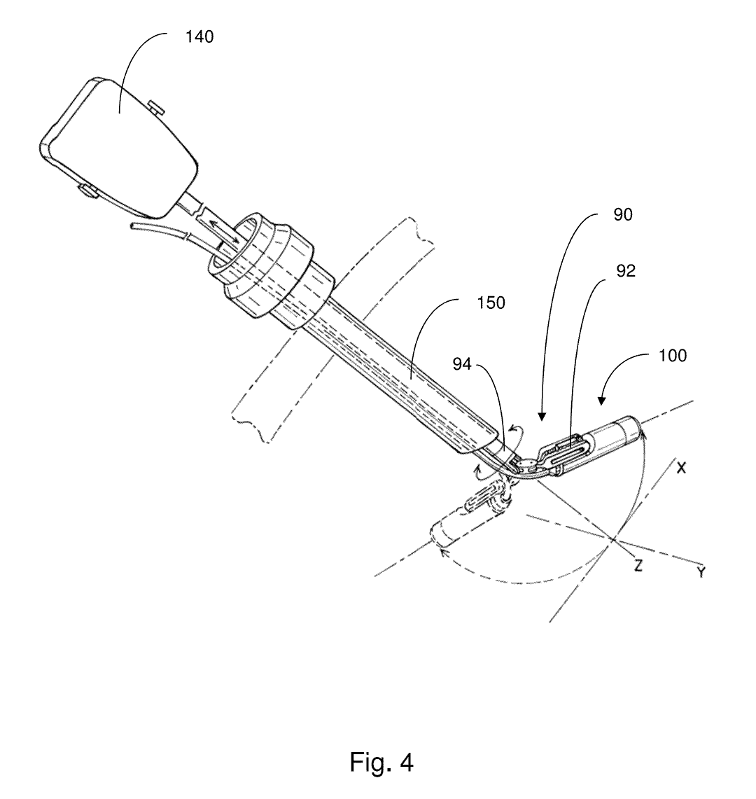

The attachment region 120 of the probe 100 allows in certain embodiments for the probe to be suitably grasped and manipulated by a tool used by a surgical robot, or by a user, for manually handling the probe 100. In some embodiments, the attachment region 120 may include a handle having a surface that suitably complements a respective surface of an appropriate grasping tool. For instance, a surgical robot (operated remotely by a user), or manual user, may operate a grasping tool to clamp down on the attachment region 120 so as to pick up and manipulate the probe 100 with a suitable amount of dexterity. FIG. 4 illustrates an example where a grasping tool attached or otherwise mounted to a surgical robot grasps the probe 100 at the attachment region 120 and, in turn, is able to move the probe in an adept manner, with wide latitude.

While the attachment region 120 is shown as having a handle with a T-shaped cross-section extending from the surface of the housing 112, it can be appreciated that such an attachment feature may have any suitable structure and will generally be configured to be compatible with or optimized for use with the gripping component/configuration of the particular surgical robot or other remote manipulator for which it is intended to be used. For example, the attachment region 120 may include a recess with protrusions jutting out within the recess that allow for a portion of an appropriate tool to be inserted therein, so as to form an interference or snap fit. Or, the attachment region 120 may include one or more adhesive and/or fastening elements that provide a manner in which the probe can be grasped or otherwise secured so as to be manipulated in a desirable manner by a respective tool, wielded by a surgical robot.

The cable 130, for embodiments that use a cable for signal transmission, is connected, either via a permanent coupling or is connectable, e.g. via an optional connector, to the radiation detector 110 and transmits the signal emitted by the radiation detecting material 150 toward the proximal end 104 of the probe. Accordingly, at the proximal end 104 of the probe 100, the cable 130 may be connected/connectable to a detector/processor for further processing the signal emitted by the radiation detecting material 150.

For instance, where the radiation detecting material 150 is a scintillator, the cable 130 may include an optical fiber arrangement that is suitable for transmitting the light emitted from the scintillator to an appropriate photosensor (e.g., photomultiplier or other device that converts the light to an electrical signal) connected at the proximal end 104. Optical fibers, in general, may be flexible, transparent fibers that are made up of high quality, thinly extruded silica (e.g., glass) and/or plastic, which are often used to form waveguides that transmit light between opposing ends of a fiber or cable. For example, optical fibers may include a light transmitting core surrounded by a particular material or cladding, often having a relatively low index of refraction, so as to maintain light within the core by total internal reflection.

In an optical fiber cable, for some embodiments, optical fibers may be surrounded by a protective sheath that allows the cable to be physically manipulated, as desired, in an appropriate environment. Such a protective sheath may include any suitable composition. For example, optical fibers in a cable may be surrounded by a sheath that includes a suitable polymer, such as polyvinyl chloride, polyethylene, polyurethane, polyethylene terephthalate, polybutylene terephthalate, polyamide, polyimide, aramid, polyethylene, or any other appropriate material.

In some embodiments, the cable 130 is flexible, so as to allow for the probe to be easily maneuvered, as appropriate, during use. For example, the cable 130 may include a flexible housing/sheath/covering material made up of a suitable thermoplastic, such as one or more of the above noted polymers, and/or other materials. Though, it can be appreciated that it is not a requirement for all embodiments of the probe to have a flexible cable. In some embodiments, the cable may be constructed of a relatively rigid material, for example, to provide added protection and/or support for the probe.

While not expressly shown in FIG. 2A, the probe 100 may be connected to, or otherwise in communication with, a signal processing apparatus and/or controller, e.g. a controller similar to controller 20 illustrated in FIG. 1. For example, the cable 130 may serve to connect the radiation detector 110 and the signal processing apparatus, located at the proximal end 104 of the probe, so as to provide a conduit for communication therebetween. In some embodiments, where the radiation detector includes a scintillator, the cable 130 may optionally be a flexible optical fiber cable that connects the scintillator to a photomultiplier (e.g., solid state photomultiplier), semiconducting light sensor (e.g., photodiode), or other suitable device for receiving and processing light.

In some cases, suitable photomultipliers may include tubes (e.g., vacuum phototubes) that are sensitive to light, including light having wavelengths in the visible and non-visible (e.g., ultraviolet, near-infrared, infrared) portions of the electromagnetic spectrum. The photomultiplier may receive the light and, in turn, generate an electrical signal that corresponds to the amount and/or intensity of received light by the photomultiplier, hence, processing the light signal.

The signal processor and/or controller may process the signal (e.g., light) received from the radiation detector and, based on the signal received (e.g., via a photomultiplier), emit another signal (e.g., electrical signal) to an information station, such as a video and/or audio device (e.g., monitor, display screen, alarm, speaker, etc.). For example, when the probe comes into close proximity with the radionuclide of interest, the radiation detector may produce a signal that the cable subsequently transmits to the signal processor and/or controller.

Based on the amount of signal produced by the radiation detector, the information station may provide visual and/or audio feedback (e.g., number, radiation detection bar, series of beeps having a particular volume or frequency) that provides an indication to a user of the amount of signal emitted from the radiation detector from exposure to the radionuclide, or the level of radiation detected from the radiation detector. From this information, coupled with knowledge of the current location of the distal end of the probe, the user or other appropriate medical personnel can make a determination as to the location of the target radionuclide.

In some embodiments, the signal processor and/or controller may be configured according to the type and amount of radiation to be detected. That is, depending on the radionuclide of interest (e.g., Technetium-99, Iodine-125, Iodine-124, etc.), the system may be set to detect certain threshold levels of signal emitted from the radiation detector. For instance, based on the system set to a particular radionuclide to be detected, when the amount of signal generated and read from the radiation detector (e.g., scintillator) exceeds the threshold (e.g., specific range of intensity of light) particular to that radionuclide, the system may provide an indication as to its presence, such as through the above described visual and/or audio feedback.

In some embodiments, the signal processor and/or controller may include a backend counting system that records and processes the information received from the radiation detector and determines the amount of ionizing radiation to which the radiation detector is exposed. This information may be stored in memory and conveyed to a user in a useful format, for example, via video and/or audio feedback, at an appropriate time.

It can be appreciated that any of the electrical signals transmitted between system components (e.g., photomultiplier, controller, display indicator, etc.) may be transmitted wirelessly, such as by implementing a dongle or other suitable wireless component at an appropriate location of the probe (e.g., proximal end) or other processing component in communication with the probe.

Accordingly, the apparatus which processes the light (or other signal) from the scintillator (or other radiation detector), and subsequently communicates with a controller, display, and/or other appropriate device, is able to be placed at a substantial distance from the distal (detection/insertion) end of the probe. That is, the signal processing portion of the probe 100, which may include a number of electrical components, remains outside the body of the patient during the medical procedure. At the same time, the radiation detecting portion (e.g., scintillator) of the probe 100, which is inserted within the body of the patient during surgery, may be free of any electrical components.

The ability for the distal end 102, or the region that is inserted within, in contact with, or is otherwise placed in substantially close proximity to the body during surgery, of embodiments of the probe 100 to be free of electrical components may confer a number of benefits. For instance, during use in surgery, electrical current or power may essentially be located away from the patient site, considerably reducing risk of damage or complications that may arise from potential problems associated with the presence of electrical components, such as current leakage, overheating, malfunction, etc. of the probe. This is in contrast to conventional detection probes that employ a radiation detecting material that generates an electrical current upon exposure to ionizing radiation. As discussed above, such probes require a number of electrical components to process the generated electrical current, often requiring the probe to incorporate a relatively large, bulky housing, to suitably contain the electrical component(s) and to protect surrounding tissue therefrom. Conventional probes that employ a photomultiplier also require electrical components for high voltage biasing, located within the handle of the probe, which often come into undesirably close proximity to the body during surgery, and contribute to the relatively large size of the probe.

For some embodiments described herein, the radiation detecting material produces light upon exposure to the ionizing radiation where an optical fiber cable is sufficient to transmit the light to a distant location. Such a stripped down arrangement allows for the distal end of the probe to be free of any electrical or power component(s).

Further, without requiring electrical components, and the protective housing, to be provided within or near the distal end 102, the probe 100 may be more easily or economically sized to fit within small spaces. For example, probes in accordance with the present disclosure may be sized for appropriate insertion into the lumen of a trocar, or other device that provides a suitable portal to the body through which medical instruments may be inserted and utilized in surgery. In contrast, typical conventional radiation detection probes such as probe 10, which require electrical components to be built therein, may be substantially larger than probes of the present disclosure and, for example, would be unable to be inserted into the lumen of a trocar having a diameter of less than or equal to 16 mm, less than or equal to 12 mm, or less than or equal to 10 mm.

In some embodiments, the lumen of the trocar through which embodiments of the probe may be inserted may have a diameter (or largest cross-sectional width/dimension where the lumen does not have a circular cross-section) of less than or equal to 16 mm, less than or equal to 14 mm, less than or equal to 12 mm, less than or equal to 10 mm, less than or equal to 8 mm, or less than or equal to 6 mm. In some embodiments, the lumen of the trocar through which the probe may be inserted may have a diameter (or largest cross-sectional width/dimension) of greater than or equal to 2 mm, greater than or equal to 4 mm, greater than or equal to 6 mm, greater than or equal to 8 mm, greater than or equal to 10 mm, or greater than or equal to 12 mm. It can be appreciated that probes in accordance with the present disclosure may be inserted into a suitable bore or lumen having a cross-sectional width/dimension falling within a range having any one of the end points noted above, or outside of these ranges.

As noted above, various components (e.g., electrical, photomultiplier, etc.) integrated into the housing of the radiation detector may be susceptible to damage or malfunction upon impact. Accordingly, a probe that lacks any such electrical components may be able to be withstand a reasonable impact (e.g., by being dropped), without being damaged.

As discussed herein, embodiments of the present disclosure may be used in cooperation with any suitable surgical robot, including appropriate accessories and instruments thereof, to perform a medical procedure. For example, suitable surgical robots with which embodiments of the present disclosure may be combined and/or incorporated include the DA VINCI® Surgical System, provided by Intuitive Surgical, amongst others.

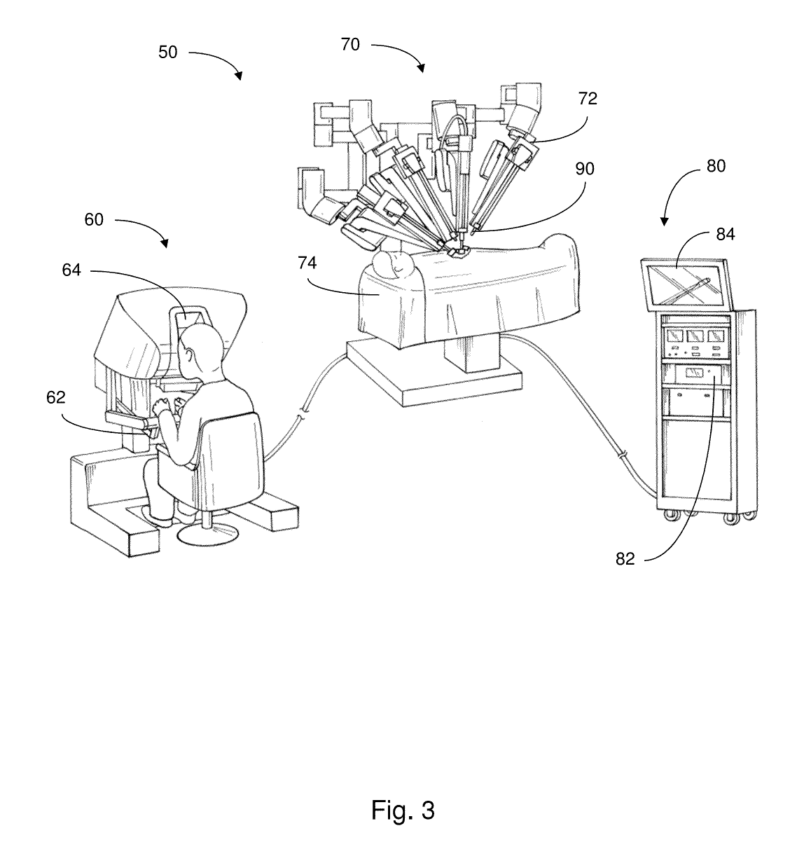

FIG. 3 shows an example of a surgical robot 50 that may employ embodiments of the radiation detection probe described herein. The surgical robot 50 includes a control station 60, a surgical station 70 and an information station 80. The control station 60 may include a number of control components 62, for example, a series of levers, handles, knobs, switches, pedals, or other suitable control features that are remotely connected to the surgical station 70, so as to allow the surgeon to control various arm components 72 thereof, for performing a medical procedure on a patient lying on a medical table 74.

The arm components 72 of the surgical station 70 may include a number of articulating joints that allow the arm(s) to be controlled with precise movement(s) over six degrees of freedom. Each of the arm components 72 may have a particular instrument 90 attached to or otherwise mounted at its distal end, which also may be controlled in a precise and appropriate manner over six degrees of freedom. The instrument(s) 90 mounted on to an arm component 72 of a surgical station 70 may include, for example, graspers, needle drivers, forceps, hooks, spatulas, scissors, scalpels, blades, shears, cautery tools, retractors, dissectors, stabilizers, staples, clip appliers, irrigators, suction tools, sealers, obturators, cannulae, obturators, insertion tools, protectors, reducers, vision equipment, or any other suitable instrument. In some embodiments, the instrument constructed for use with a surgical robot may be able to suitably grasp and handle a radiation detection probe, in accordance with the present disclosure, or may itself incorporate the radiation detection probe.

In some embodiments, the instrument 90 includes a manipulating tool, an elongated shaft and a control module. The control module may be interfaced, at a proximal end, with an articulating arm component 72 of the surgical station 70. For example, the control module may have a number of knobs, buttons, levers, etc. that are operable by the articulating arm component 72, for appropriately controlling the manipulating tool. The elongated shaft may extend distally from the control module, and the manipulating tool may be located at the distal end of the instrument.

The control component(s) 62 of the control station 60 may be operated by an appropriately trained surgeon, so as to control movement of the arm component(s) 72 of the surgical station 70 and the corresponding instrument(s) 90 mounted thereto. For instance, an instrument including a pair of forceps at its distal end, and mounted to an arm component 72 of the surgical station 70, may be suitably moved and controlled via the control module. That is, upon establishment of an appropriate interface between the control module and the arm component 72, the surgeon may remotely direct movement of the forceps in a desired manner.

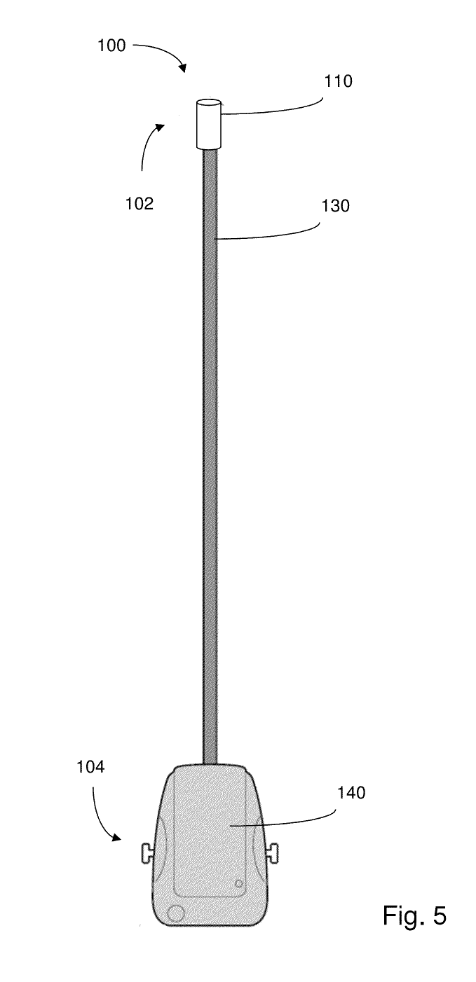

As shown further below in FIG. 5, an instrument may include a probe 100 for detecting radiation, such as an embodiment of probes described herein. The instrument incorporating the probe 100 may be interfaced with the surgical station via an appropriate control module, enabling the surgeon to control movement of the probe itself in a suitable manner, via the control interface.

To sense/observe whatever activity that may be occurring at the surgical station 70, the control station 60 may also provide a number of other components that provide the user with sensory feedback. For example, the control station 60 may include a viewfinder 64 into which the surgeon may peer, so as to observe images/video of the surgery on a monitor, sent from the surgical station 70. For instance, a camera may be mounted on the distal end of one of the articulating arm components 72 of the surgical station 70 so as to provide real-time video to the operating surgeon via the monitor provided by the viewfinder 64. As a result, the surgeon is able to perform the surgery remotely from the control station 60, by manipulating the instrument(s) mounted to the various components 72 of the surgical station 70.

As noted above, the surgical robot 50 further may further include an information station 80 that may provide visual and/or audio information regarding the status of the surgery. For example, the information station 80 may include a controller 82 for sending and receiving signals to and from the control station 60 and/or the surgical station 70, and therebetween. The controller 82 may communicate with a display 84 which provides a monitor for those nearby to view what is occurring in the surgery and how it is progressing. The information station 80 may provide any appropriate information, such as patient vitals and/or signals indicating the status of one or more regions of the body. In accordance with the present disclosure, the information station 80 may also provide an indication, during use within the body of a patient, as to whether the probe is presently exposed to ionizing radiation, sufficient to provide a determination of the location of a radionuclide within the body.

FIG. 4 depicts a medical procedure where an embodiment of a probe 100, shown in FIGS. 2A-2B, is used in cooperation with a suitable surgical robot. In this example, the arm of a surgical robot wields a grasping tool 90 having forceps 92, a shaft 94 and a control module (not shown in the figures). The shaft 94 provides an elongated interconnection between the forceps 92, located at the distal end of the tool, and the control module, located at the proximal end of the tool. The control module of the tool 90 is interfaced with an appropriate articulating arm component 72 of the surgical robot, such that the surgeon is able to move and manipulate the forceps 92 with precise articulation along six degrees of freedom, remotely via the control station 60.

The tool shown in the embodiment of FIG. 4 is similar to the ENDOWRIST® instrument implementing Cadiere Forceps, provided by Intuitive Surgical. Here, the slot of the forceps 92 structurally complements the handle 120 of the probe 100 to form a suitable attachment therebetween. Accordingly, the forceps 92 are able to securely grasp and manipulate the probe 100 with minimal concern that the probe will be mishandled or dropped. In contrast, the conventional probe 10 depicted in FIG. 1 has no such attachment feature that accommodates grasping by forceps employed by a surgical robot.

As discussed above, embodiments of probes in accordance with the present disclosure may be sized so as to be able to fit through the lumen of a suitable trocar, which is used during surgery as a portal through which surgical instruments may be deployed. FIG. 4 further shows the arm 90 and the probe 100 disposed within the lumen of a 8 mm trocar 150.

Not only are probes described herein able in certain embodiments to be inserted through a relatively small lumen of a trocar, by using high-precision surgical robots, the probes are also in certain embodiments able to move according to complex patterns along six degrees of freedom, movement that is generally not possible using traditional laparoscopic techniques.

It can be appreciated that other configurations and arrangements of the probe may be used, in cooperation with the surgical robot. For example, probes in accordance with the present disclosure may grasped and manipulated by instruments other than those discussed herein.

In some embodiments, the probe may be integrated together with the instrument, for use with and control by the surgical robot. That is, the surgical robot may be fitted or otherwise provided with an instrument that itself includes a probe in accordance with aspects of the present disclosure. Accordingly, for some embodiments, a separate instrument having a grasping tool (e.g., forceps 92) would not be necessary for suitable use of the probe during the medical procedure.

FIG. 5 shows an illustrative embodiment of a probe 100 that may be used as an instrument mounted directly on to an articulating arm component of a surgical robot. The probe 100 has a distal end 102 that is inserted into the body of the patient during surgery, and a proximal end 104 that remains outside of the body of the patient during surgery. The probe 100 includes a radiation detector 110 including a suitable radiation detecting material (e.g., scintillator), a cable 130 (e.g., optical fiber cable) and a control module 140. Similar to that described above with respect to the embodiments of FIGS. 3-4, the control module 140 may be interfaced with an arm component 72 of the surgical station 70, for appropriate manipulation of the probe 100 by a remote operator. Accordingly, the probe shown in FIG. 5 does not need to be grasped by another instrument (e.g., forceps) mounted to the surgical robot.

As discussed herein, embodiments of the present disclosure may be used to detect the location of one or more cancerous regions within the body of a patient. When fighting cancer, for a proper treatment plan to be devised, it is important to know whether the cancer has spread to other parts of the body, such as the lymphatic system. When assessing whether a cancer has spread to the lymphatic system, physicians often look for the sentinel lymph node, which is the first lymph node or group of nodes through which the cancerous tissue/cells drain(s). If the sentinel lymph node is not found to contain cancer, then it is likely that the cancer has not spread to other areas of the body.

In preparation for a surgical operation where a probe in accordance with the present disclosure may be used, the patient may be injected with an appropriate radionuclide, such as Technetium-99 (Tc99), Iodine-124 (I124), Iodine-125 (I125), or another radioactive isotope or seed suitable for use in medical applications. The type of radionuclide injected into a patient will depend on the particular treatment or diagnostic plan for the patient. For instance, the power produced from a Tc99 radioactive seed may relatively strong (e.g., up to 150 keV) as compared to the power produced from a I125 radioactive seed. So, a Tc99 seed may be more easily detectable by a radiation detection probe than, for example, a I125 seed. Though, a I125 radioactive seed may last for up to 90 days, and so this type of radionuclide may be used for relatively longer term monitoring than, for example, a Tc99 seed.

Employing the surgical robot, grasping, or fitted with, a radiation detection probe in accordance with certain embodiments of the present disclosure, and other instruments appropriate for cancer surgery, the operator would remotely control the surgical robot to manipulate the probe. Accordingly, the operator is able to inspect specific regions of the body and determine whether the injected radionuclide is present at those regions. By detecting the presence of the radionuclide, the surgeon is able to determine what regions within the body are cancerous.

### Example

The following provides an illustrative example of a probe in accordance with the present disclosure. In this example, the probe is designed to detect gamma radiation emitted from a radionuclide tracer previously injected at a cancerous region of the body, having an energy of less than 600 keV. The probe is constructed to have a distal end and a proximal end where, during surgery, the distal end of the probe is inserted into the body of the patient, while the proximal end of the probe remains outside of the body. This example is depicted in FIGS. 2A-2B, and use of this example is further depicted in FIG. 4.

In this example, the probe includes a scintillator located at a distal end. The scintillator is made up of a BGO crystal, which emits light upon exposure to gamma radiation. Depending on the level of the gamma radiation to which the scintillator is exposed, the scintillator will, in turn, emit light having a generally proportionate level of intensity. The scintillator is surrounded by a tungsten collimator for shielding energy emitted from the side of the scintillator. And, the collimator is surrounded by a stainless steel housing, for protecting the scintillator housed therein.

The probe further includes a flexible optical fiber cable that extends in a proximal direction from the scintillator. The flexible optical fiber cable is coupled at its distal end to the scintillator, and transmits light emitted from the scintillator therethrough. The flexible optical fiber cable is further coupled at its proximal end to a solid state photomultiplier, which receives the transmitted light from the scintillator and generates an electrical current, proportionate to the intensity of light received.

The electrical current generated from the photomultiplier is further processed by a controller that is configured to provide feedback information (e.g., visual and/or audio information) to a user as to the amount of light emitted from the scintillator from exposure to the gamma radiation. From this information, the user or other medical personnel can make a determination as to the location of the gamma ray emitting tracer and, hence, the location of a potentially cancerous region.

In this example, the portion of the apparatus that processes the light emitted from the scintillator ultimately resulting in feedback information communicated to a user as to the potential location of a cancerous region, is able to be kept at a suitable distance away from the distal, detection/insertion end of the probe. For example, the photomultiplier, electrical/power components, or other relatively large or bulky components associated with the system remains outside the body of the patient and a suitable distance afar during the medical procedure.

With various components of the system remaining at a suitable distance away from the insertion end of the probe, the probe is able to fit within small spaces, such as the lumen of a relatively small trocar having a diameter of less than or equal to 16 mm, less than or equal to 12 mm, or less than or equal to 10 mm. In addition, without various electrical and/or power components integrated at or near the insertion end, when dropped or subject to reasonable impact, the probe is less likely to incur damage.

The probe has a handle with a T-shaped cross-section extending from the surface of the housing. This handle may be grasped and manipulated with dexterity by the Cadiere forcep, mounted on to the DA VINCI® Surgical System, provided by Intuitive Surgical. Because the optical fiber cable is flexible, the probe may be easily maneuvered, as appropriate, during use.

Any of the above aspects may be employed in any suitable combination as the present invention is not limited in this respect. Also, any or all of the above aspects may be employed in cancer detection or other medical applications; however, the present invention is not limited in this respect, as aspects of the present disclosure may be employed to suit other applications. While various embodiments of probes described herein may be configured for passage through a suitably sized trocar, certain embodiments of probes may be used or adapted to be used in other procedures (e.g., laparoscopic) that do not involve surgical robots.

Having thus described several aspects of at least one embodiment of this invention, it is to be appreciated various alterations, modifications, and improvements will readily occur to those skilled in the art. Such alterations, modification, and improvements are intended to be part of this disclosure, and are intended to be within the spirit and scope of the invention. Accordingly, the foregoing description and drawings are by way of example only.

What is claimed is:

1. A probe having a distal end and a proximal end, configured for use in a medical procedure, the probe comprising:
a scintillator constructed to be able to be inserted into a body of a patient by passing through the lumen of a trocar, wherein the lumen has a largest cross-sectional dimension not exceeding 16 mm, and wherein the scintillator is adapted to emit a signal upon exposure to a radionuclide; and
an optical fiber connected to the scintillator, for transmitting the signal emitted from the scintillator.

2. The probe of claim 1, wherein the scintillator is located at the distal end of the probe, and wherein the distal end is arranged to be inserted into the body of the patient during the medical procedure.

3. The probe of claim 2, wherein the distal end of the probe that is inserted into the body of the patient during the medical procedure is free of any electrical component.

4. The probe of claim 1, wherein the optical fiber is flexible.

5. The probe of claim 1, wherein the largest cross-sectional dimension of the lumen of the trocar does not exceed 12 mm.

6. The probe of claim 1, wherein the largest cross-sectional dimension of the lumen of the trocar does not exceed 10 mm.

7. The probe of claim 1, wherein the scintillator includes bismuth germinate.

8. The probe of claim 1, wherein the scintillator is adapted to emit light upon exposure to gamma rays originating from the radionuclide.

9. The probe of claim 1, wherein the probe comprises or is connectable to a signal processor and/or controller located at the proximal end of the probe, the signal processor and/or controller being configured to process the signal emitted from the scintillator and to remain outside the body of the patient during the medical procedure.

10. The probe of claim 9, wherein the signal processor and/or controller includes a photomultiplier.

11. A probe having a distal end and a proximal end, configured for use in a medical procedure, the probe comprising:
a radiation detector located at the distal end, adapted to emit a signal upon exposure to a radionuclide, wherein the radiation detector is constructed to be able to be inserted into a body of a patient by passing through the lumen of a trocar, wherein the lumen has a largest cross-sectional dimension not exceeding 16 mm; and
a flexible cable connected at its distal end to the radiation detector and connected or connectable at its proximal end to a signal processor and/or controller, the flexible cable configured to transmit the signal emitted from the radiation detector and/or information generated from the signal to the signal processor and/or controller.

12. The probe of claim 11, wherein the distal end of the probe is arranged to be inserted into a body of a patient during the medical procedure.

13. The probe of claim 12, wherein the distal end of the probe that is inserted into the body of the patient during the medical procedure is free of any electrical component.

14. The probe of claim 11, wherein the radiation detector is constructed to be able to be inserted into a body of a patient by passing through the lumen of a trocar, wherein the lumen has a largest cross-sectional dimension not exceeding 12 mm.

15. The probe of claim 11, wherein the radiation detector includes a scintillator.

16. The probe of claim 11, wherein the flexible cable includes an optical fiber.

17. The probe of claim 11, wherein the signal processor and/or controller is configured to process the signal emitted from the radiation detector and to remain outside the body of the patient during the medical procedure.

18. A probe system comprising a probe having a distal end and a proximal end, configured for use in a medical procedure, the probe system comprising:
a radiation detector located at the distal end of the probe, adapted to emit a signal upon exposure to a radiation source and arranged to be inserted within the body of a patient during the medical procedure; and
a signal processor and/or controller located at or connected to the proximal end of the probe, configured to process the signal emitted from the radiation detector and to remain outside the body of the patient during the medical procedure,
wherein the distal end of the probe that is inserted into the body of the patient during the medical procedure is free of any electrical component.

19. The probe system of claim 18, wherein the radiation detector is constructed to be able to be inserted into a body of a patient by passing through the lumen of a trocar, wherein the lumen has a largest cross-sectional dimension not exceeding 16 mm.

20. The probe system of claim 18, wherein the radiation detector includes a scintillator.

21. The probe system of claim 18, further comprising a flexible cable configured to transmit the signal emitted from the radiation detector and/or information generated from the signal to the signal processor and/or controller.

22. The probe system of claim 18, wherein the signal processor and/or controller is configured to detect an amount of signal emitted from the radiation detector.

23. The probe system of claim 22, wherein the signal processor and/or controller includes a photomultiplier.

24. The probe system of claim 22, wherein the signal processor and/or controller is configured to transmit a signal indicating the amount of signal emitted from the radiation detector to a display and/or audio indicator.

25. The probe system of claim 24, wherein the signal processor and/or controller is configured for wireless communication with the display and/or audio indicator.

26. A system for performing a medical procedure, the system comprising:
a surgical robot configured to perform remote surgery; and
a probe comprising a grasping feature shaped and arranged to facilitate being grasped and manipulated by an arm of the surgical robot, wherein the probe is configured to detect a radiation source located within a body of a patient.

27. The system of claim 26, wherein the grasping feature includes a handle having a surface that complements a surface of the arm of the surgical robot.

28. The system of claim 26, wherein the probe is configured to identify of a potentially cancerous region of the body of the patient.

29. The system of claim 26, wherein the probe includes a radiation detector adapted to emit a signal upon exposure to a radionuclide.

30. The system of claim 29, further comprising a signal processor and/or controller configured to detect an amount of signal emitted from the radiation detector.

31. The system of claim 30, further comprising a display and/or audio indicator, wherein the signal processor and/or controller is configured to transmit a signal indicating the amount of signal emitted from the radiation detector to the display and/or audio indicator.

32. The system of claim 31, wherein the signal processor and/or controller is configured for wireless communication with the display and/or audio indicator.

33. A method for performing a medical procedure, the method comprising:
grasping a probe using a surgical robot, wherein the probe is configured to detect a radiation source located within a body of a patient; and
manipulating the probe within the body of the patient with the surgical robot to detect the radiation source within the patient.

34. The method of claim 33, wherein grasping the probe using the surgical robot includes manipulating an arm of the surgical robot to grasp a handle of the probe having a surface that complements a surface of the arm of the surgical robot.

35. The method of claim 33, wherein manipulating the probe to detect the radiation source includes identifying a potentially cancerous region of the body of the patient.

36. The method of claim 33, wherein manipulating the probe to detect the radiation source includes detecting a presence of a radionuclide by a radiation detector adapted to emit a signal upon exposure to the radionuclide.

37. The method of claim 36, further comprising detecting an amount of signal emitted from the radiation detector due to exposure to the radionuclide.

38. The method of claim 37, further comprising transmitting a signal indicating the amount of signal emitted from the radiation detector to a display and/or audio indicator for informing a user of the amount of signal emitted from the radiation detector due to exposure to the radionuclide.

39. The method of claim 38, wherein transmitting the signal indicating the amount of signal emitted from the radiation detector to the display and/or audio indicator includes wireless communication with the display and/or audio indicator.

40. A surgical device configured for use in a medical procedure comprising:
a radiation detector adapted to emit a signal upon exposure to a radionuclide, wherein the radiation detector is constructed to be able to be inserted into a body of a patient by passing through the lumen of a trocar, wherein the lumen has a largest cross-sectional dimension not exceeding 16 mm; and
wherein the radiation detector comprises a connector configured for connection to a flexible cable for transmitting the signal emitted from the radiation detector and/or information generated from the signal to a signal processor and/or controller, and/or comprises a transmitter configured to wirelessly transmit the signal emitted from the radiation detector and/or information generated from the signal to a signal processor and/or controller.

41. The surgical device of claim 40, wherein the radiation detector comprises a scintillator; and wherein the radiation detector comprises a connector configured for connection to an optical fiber for transmitting the signal emitted from the scintillator.

42. An apparatus for use with a surgical robot, the apparatus comprising:
a probe comprising a grasping feature shaped and arranged to facilitate being grasped and manipulated by an arm of the surgical robot, wherein the probe is configured to detect a radiation source located within a body of a patient.

43. The apparatus of claim 42, wherein the grasping feature includes a handle having a surface that complements a surface of the arm of the surgical robot.

44. The apparatus of claim 42, wherein the probe is configured to identify of a potentially cancerous region of the body of the patient.

45. The apparatus of claim 42, wherein the probe includes a radiation detector adapted to emit a signal upon exposure to a radionuclide.

46. The apparatus of claim 45, further comprising a signal processor and/or controller configured to detect an amount of signal emitted from the radiation detector.

47. The apparatus of claim 46, further comprising a display and/or audio indicator, wherein the signal processor and/or controller is configured to transmit a signal indicating the amount of signal emitted from the radiation detector to the display and/or audio indicator.

48. The apparatus of claim 47, wherein the signal processor and/or controller is configured for wireless communication with the display and/or audio indicator.

49. The apparatus of claim 42, further comprising the surgical robot.

50. An apparatus having a distal end and a proximal end, for use with a surgical robot, the apparatus comprising:
a handle having a surface that complements a surface of the arm of the surgical robot to facilitate being grasped and manipulated by the arm of the surgical robot;
a scintillator located at the distal end, constructed to be able to be inserted into a body of a patient by passing through the lumen of a trocar, wherein the lumen has a largest cross-sectional dimension not exceeding 12 mm, and wherein the scintillator is adapted to emit an optical signal upon exposure to gamma radiation;
a photomultiplier located at the proximal end, configured to receive the optical signal emitted from the scintillator and generate an electrical current based on an amount of optical signal received from the scintillator, the photomultiplier configured to remain outside the body of the patient during the medical procedure;
a flexible optical fiber cable connected at its distal end to the scintillator and connected or connectable at its proximal end to the photomultiplier, the flexible optical fiber cable configured to transmit the optical signal emitted from the scintillator to the photomultiplier; and
wherein the distal end of the apparatus that is inserted into the body of the patient during the medical procedure is free of any electrical component.

51. The apparatus of claim 50, further comprising the surgical robot.

52. The apparatus of claim 51, wherein the surgical robot includes a DA VINCI® Surgical System.
